(19) United States
(12) Patent Application Publication
(10) Pub. No.: US 2017/0189305 A1
(43) Pub. Date: Jul. 6, 2017

(54) EMULSIONS CONTAINING FILM FORMING DISPERSION OF PARTICLES IN AQUEOUS PHASE AND HYDROPHOBIC FILLER

(71) Applicant: L'ORÉAL, Paris (FR)

(72) Inventors: Mikhail Motornov, Clark, NJ (US); Angeles Fonolla-Moreno, Scotch Plains, NJ (US)

(73) Assignee: L'ORÉAL, Paris (FR)

(21) Appl. No.: 14/985,129

(22) Filed: Dec. 30, 2015

(51) Int. Cl. A61K 8/81; A61Q 1/02; A61K 8/06; A61K 8/73; A61K 8/34; A61K 8/368; A61K 8/60; A61K 8/893; A61K 8/86; A61K 8/92; A61K 8/58; A61K 8/96; A61K 8/25; A61K 8/19; A61K 8/29; A61Q 1/10

(52) U.S. Cl. CPC A61K 8/8182; A61Q 1/10; A61Q 1/02; A61K 8/06; A61K 8/735; A61K 8/345; A61K 8/34; A61K 8/368; A61K 8/602; A61K 8/893; A61K 8/86; A61K 8/8111; A61K 8/927; A61K 8/585; A61K 8/965; A61K 8/25; A61K 8/19; A61K 8/29; A61K 2800/43; A61K 2800/592

(57) ABSTRACT

The invention relates to a composition, especially a cosmetic emulsion composition, comprising at least one dispersion of film forming particles in aqueous phase and at least one hydrophobic filler, as well as to methods of using such compositions.

### FIELD OF THE INVENTION

The present invention relates to emulsions comprising at least one dispersion of film forming particles in aqueous phase and at least one hydrophobic filler.

### DISCUSSION OF THE BACKGROUND

Currently available cosmetic products such as eye shadows, foundations, etc. typically contain volatile organic solvents, such as isododecane and a significant amount of film formers such as latex or silicone resins, as these components are known to assist in imparting long wear properties to the compositions. Due to the high level of film formers, these compositions may have a tacky or sticky feel. Also, the use of volatile organic solvents makes removal difficult and is drying to the skin. Additionally, many such compositions which are eyeshadows result increasing over time. Finally, it is difficult to formulate stable compositions containing silicone resins and a significant amount of water.

There remains a need for improved cosmetic compositions having improved cosmetic properties, particularly eyeshadows, foundations, eyeliners, mascaras, concealers and blushes, which are long wearing, transfer-resistant and/or possess good comfort and/or removability properties.

Accordingly, one aspect of the present invention is a care and/or makeup and/or treatment composition for keratinous material which has good cosmetic properties such as, for example, it is long wearing, creasing-resistant and/or transfer-resistant, and/or possesses good comfort and/or removability properties.

### SUMMARY OF THE INVENTION

The present invention relates to emulsions comprising at least one dispersion of film forming particles in aqueous phase and at least one hydrophobic filler.

The present invention also relates to a reaction product of at least one dispersion of film forming particles in aqueous phase and at least one hydrophobic filler.

The present invention also relates to emulsions comprising a reaction product of at least one dispersion of film forming particles in aqueous phase and at least one hydrophobic filler.

The present invention also relates to methods of treating, caring for and/or making up keratinous material (for example, eyelashes) by applying emulsions of the present invention comprising at least one dispersion of film forming particles in aqueous phase and at least one hydrophobic filler, to the keratinous material in an amount sufficient to treat, care for and/or make up the keratinous material. Preferably, the emulsion comprises a reaction product of at least one dispersion of film forming particles in aqueous phase and at least one hydrophobic filler.

The present invention also relates to methods of enhancing the appearance of keratinous material (for example, eyelashes) by applying emulsions of the present invention comprising at least one dispersion of film forming particles in aqueous phase and at least one hydrophobic filler, to the keratinous material in an amount sufficient to enhance the appearance of the keratinous material. Preferably, the emulsion comprises a reaction product of at least one dispersion of film forming particles in aqueous phase and at least one hydrophobic filler.

The present invention also relates to methods of reducing tackiness of a film formed by at least one dispersion of film forming particles in aqueous phase comprising combining at least one hydrophobic filler with the at least one dispersion of film forming particles in aqueous phase in an emulsion in an amount sufficient to reduce the tackiness of a film formed by the at least one dispersion of film forming particles in aqueous phase. Preferably, the at least one dispersion of film forming particles in aqueous phase and at least one hydrophobic filler are combined to form a reaction product.

The present invention further relates to kits containing a n emulsion of the present invention comprising at least one dispersion of film forming particles in aqueous phase and at least one hydrophobic filler. Preferably, the emulsion comprises a reaction product of at least one dispersion of film forming particles in aqueous phase and at least one hydrophobic filler.

It is to be understood that both the foregoing general description and the following detailed description are exemplary and explanatory only, and are not restrictive of the invention.

### DETAILED DESCRIPTION OF THE INVENTION

In the following description of the invention and the claims appended hereto, it is to be understood that the terms used have their ordinary and accustomed meanings in the art, unless otherwise specified.

“About” as used herein means within 10% of the indicated number (e.g. “about 10%” means 9%-11% and “about 2%” means 1.8%-2.2%).

“A” or “an” as used herein means “at least one.”

As used herein, all ranges provided are meant to include every specific range within, and combination of subranges between, the given ranges. Thus, a range from 1-5, includes specifically 1, 2, 3, 4 and 5, as well as subranges such as and 2-5, 3-5, 2-3, 2-4, 1-4, etc.

“Film former”, “film-forming polymer” or “film forming agent” or “co-film former” as used herein means a polymer or resin that leaves a film on the substrate to which it is applied, for example, after a solvent accompanying the film former has evaporated, absorbed into and/or dissipated on the substrate.

“Free” or “devoid” of as it is used herein means that while it is preferred that no amount of the specific component be present in the composition, it is possible to have very small amounts of it in the compositions of the invention provided that these amounts do not materially affect at least one, preferably most, of the advantageous properties of the compositions of the invention. Thus, for example, “free of solvents” means that non-aqueous solvents are preferably omitted (that is 0% by weight), but can be present in the composition at an amount of less than about 0.25% by weight, typically less than about 0.1% by weight, typically less than about 0.05% by weight, based on the total weight of the composition.

“Makeup Result” as used herein, refers to compositions where color remains the same or substantially the same as at the time of application, as viewed by the naked eye, after an extended period of time. “Makeup Result” may be evaluated by evaluating long wear properties by any method known in the art for evaluating such properties. For example, long wear may be evaluated by a test involving the application of a composition to keratin materials such as eyelashes and evaluating the color of the composition after an extended period of time. For example, the color of a composition may be evaluated immediately following application to keratin materials such as eyelashes and these characteristics may then be re-evaluated and compared after a certain amount of time. Further, these characteristics may be evaluated with respect to other compositions, such as commercially available compositions.

“Making up” as used herein means to provide decoration (for example, color) to keratin materials such as the eyelashes.

“Protecting” as used herein means to inhibit damage to keratin materials such as the eyelashes by providing a protective layer on the keratin materials.

“Substituted” as used herein, means comprising at least one substituent. Non-limiting examples of substituents for substitution include atoms, such as oxygen atoms and nitrogen atoms, as well as functional groups, such as hydroxyl groups, ether groups, alkoxy groups, acyloxyalkyl groups, oxyalkylene groups, polyoxyalkylene groups, carboxylic acid groups, amine groups, acylamino groups, amide groups, halogen containing groups, ester groups, thiol groups, sulphonate groups, thiosulphate groups, siloxane groups, and polysiloxane groups. The substituent(s) may be further substituted.

“Water resistance” as used herein, means resistance of a material (substance) to the penetration of water, which may cause degradation of that material. The method implemented if assessment of this invention is further disclosed.

“Easy removal” means the composition may be substantially removed with a non-harsh remover, such as soap and water, and without excessive rubbing.

“Transfer resistance” as used herein refers to the quality exhibited by compositions that are not readily removed by contact with another material, such as, for example, a glass, an item of clothing or the skin, for example, when eating or drinking. Transfer resistance may be evaluated by any method known in the art for evaluating such. For example, transfer resistance of a composition may be evaluated by a “kiss” test. The “kiss” test may involve application of the composition to human keratin material such as hair, skin or lips followed by rubbing a material, for example, a sheet of paper, against the hair, skin or lips after expiration of a certain amount of time following application, such as 2 minutes after application. Similarly, transfer resistance of a composition may be evaluated by the amount of product transferred from a wearer to any other substrate, such as transfer from the hair, skin or lips of an individual to a collar when putting on clothing after the expiration of a certain amount of time following application of the composition to the hair, skin or lips. The amount of composition transferred to the substrate (e.g., collar, or paper) may then be evaluated and compared. For example, a composition may be transfer resistant if a majority of the product is left on the wearer's hair, skin or lips. Further, the amount transferred may be compared with that transferred by other compositions, such as commercially available compositions. In a preferred embodiment of the present invention, little or no composition is transferred to the substrate from the hair, skin or lips.

“Aqueous phase” means the phase comprising water as well as such substances of a formulation which, due to their hydrophilic character, can be mixed and/or dissolved and/ or dispersed in water. The aqueous phase of the composition according to the invention is preferably a continuous aqueous phase.

“Emulsifier” or “surfactant” is a term of art that is well known to those skilled in the art. See, e.g. http://pharmlabs.unc.edu/labs/emulsions/agents.htm. It is a compound that has a hydrophilic part and a lipophilic part (“amphiphilic) and facilitates the dispersion of two mutually insoluble phases, in this case the oil and water phases, assisting in the formation of an emulsion. Such compounds preferably do not have an overall electric charge in their working environment (are “non-ionic”).

“Oil phase” or “oily phase” means the phase containing the lipophilic, non-ionic compounds that are liquid at room temperature (25° C.). These compounds include one or more mutually compatible non-aqueous fatty substances that are liquid at room temperature, for example organic solvents and oils (“liquid fatty substances”) as herein described, and any lipophilic additive that may be present.

“Tackiness” as used herein refers to the adhesion between two substances. For example, the more tackiness there is between two substances, the more adhesion there is between the substances. To quantify “tackiness,” it is useful to determine the “work of adhesion” as defined by IUPAC associated with the two substances. Generally speaking, the work of adhesion measures the amount of work necessary to separate two substances. Thus, the greater the work of adhesion associated with two substances, the greater the adhesion there is between the substances, meaning the greater the tackiness is between the two substances.

Work of adhesion and, thus, tackiness, can be quantified using acceptable techniques and methods generally used to measure adhesion, and is typically reported in units of force time (for example, gram seconds (“g s”)). For example, the TA-XT2 from Stable Micro Systems, Ltd. can be used to determine adhesion following the procedures set forth in the TA-XT2 Application Study (ref: MATI/PO.25), revised January 2000, the entire contents of which are hereby incorporated by reference. According to this method, desirable values for work of adhesion for substantially non-tacky substances include less than about 0.5 g s, less than about 0.4 g s, less than about 0.3 g s and less than about 0.2 g s. As known in the art, other similar methods can be used on other similar analytical devices to determine adhesion.

The compositions and methods of the present invention can comprise, consist of, or consist essentially of the essential elements and limitations of the invention described herein, as well as any additional or optional ingredients, components, or limitations described herein or otherwise useful.

Referred to herein are trade names for materials including, but not limited to polymers and optional components. The inventors herein do not intend to be limited by materials described and referenced by a certain trade name. Equivalent materials (e.g., those obtained from a different source under a different name or catalog (reference) number) to those referenced by trade name may be substituted and utilized in the methods described and claimed herein.

All percentages and ratios are calculated by weight unless otherwise indicated. All percentages are calculated based on the total weight of a composition unless otherwise indicated. All component or composition levels are in reference to the active level of that component or composition, and are exclusive of impurities, for example, residual solvents or by-products, which may be present in commercially available sources.

Dispersion of Film Forming Particles

According to the present invention, compositions (that is, emulsions) comprising at least one dispersion of film forming particles in aqueous phase are provided. The dispersion of film forming particles in aqueous phase is more generally known as latex.

Suitable polymers for the film-forming particles that may be used in the compositions of the present invention include, but are not limited to, synthetic polymers, free-radical type or polycondensate type polymers, polymers of natural origin, and mixtures thereof.

Preferably, the polymers for the film-forming particles may be selected from vinyl (co)polymers, (meth)acrylic (co)polymers, urethanes (co)polymers, and mixtures thereof. Advantageously, the polymer for the film-forming particles is selected from a styrene-(meth)acrylic and (meth)acrylic copolymer, a vinyl acetate and (meth)acrylic copolymer, and mixtures thereof.

Polymers for the film-forming particles of the free-radical type may be chosen, for example, from vinyl polymers or copolymers, such as acrylic polymers.

Vinyl film-forming polymers can result from the polymerization of monomers comprising at least one ethylenic unsaturation and at least one acidic group and/or esters of these acidic monomers and/or amides of these acidic monomers. Monomers comprising at least one acid group which may be used include, for example, α,β-ethylenic unsaturated carboxylic acids such as acrylic acid, methacrylic acid, crotonic acid, maleic acid or itaconic acid. (Meth)acrylic acid and crotonic acid are, for example, used. Preferably, (meth)acrylic acid is used.

The esters of acidic monomers can be chosen, for example, from (meth)acrylic acid esters (also known as (meth)acrylates), such as (meth)acrylates of an alkyl, for example, a C₁-C₃₀ alkyl, such as a C1-C20 alkyl, (meth)acrylates of an aryl, such as a C₆-C₁₀ aryl, and (meth)acrylates of a hydroxyalkyl, such as a C₂-C₆ hydroxyalkyl. Among the alkyl (meth)acrylates that may be mentioned, examples include methyl methacrylate, ethyl methacrylate, butyl methacrylate, isobutyl methacrylate, 2-ethylhexyl methacrylate, lauryl methacrylate and cyclohexyl methacrylate. Among the hydroxyalkyl (meth)acrylates that may be mentioned, examples include hydroxyethyl acrylate, 2-hydroxypropyl acrylate, hydroxyethyl methacrylate and 2-hydroxypropyl methacrylate. Among the aryl (meth)acrylates that may be mentioned, examples include benzyl acrylate and phenyl acrylate. The (meth)acrylic acid esters that may be used are, for example, alkyl (meth)acrylates.

The alkyl group of the esters may be substituted. For example, the alkyl group of the esters may be either fluorinated or perfluorinated, i.e., some or all of the hydrogen atoms of the alkyl group are substituted with fluorine atoms. Further, examples of amides of the acid monomers that may be mentioned include (meth)acrylamides, such as N-alkyl(meth)acrylamides, for example, of a C₂-C₁₂ alkyl. Among the N-alkyl(meth)acrylamides that may be mentioned, examples include N-ethylacrylamide, N-t-butylacrylamide, N-t-octylacrylamide and N-undecylacrylamide.

The vinyl film-forming polymers may also result from the homopolymerization or copolymerization of monomers chosen from vinyl esters and styrene monomers. For example, these monomers may be polymerized with acid monomers and/or esters thereof and/or amides thereof, such as those mentioned above. Examples of vinyl esters that may be mentioned include vinyl acetate, vinyl neodecanoate, vinyl pivalate, vinyl benzoate and vinyl t-butylbenzoate. Styrene monomers that may be mentioned include styrene and α-methylstyrene.

Among the film-forming polycondensates that may be mentioned, examples include polyurethanes, polyesters, polyesteramides, polyamides, epoxyester resins and polyureas.

The polyurethanes may be chosen from anionic, cationic, nonionic or amphoteric polyurethanes, polyurethane-acrylics, polyurethane-polyvinylpyrrolidones, polyester-polyurethanes, polyether-polyurethanes, polyureas and polyurea-polyurethanes, and mixtures thereof.

The polyesters may be obtained, in a known manner, by polycondensation of dicarboxylic acids with polyols, such as diols.

The dicarboxylic acid may be aliphatic, alicyclic or aromatic. Examples of such acids that may be mentioned include: oxalic acid, malonic acid, dimethylmalonic acid, succinic acid, glutaric acid, adipic acid, pimelic acid, 2,2-dimethylglutaric acid, azelaic acid, suberic acid, sebacic acid, fumaric acid, maleic acid, itaconic acid, phthalic acid, dodecanedioic acid, 1,3-cyclohexanedicarboxylic acid, 1,4-cyclohexanedicarboxylic acid, isophthalic acid, terephthalic acid, 2,5-norbornanedicarboxylic acid, diglycolic acid, thiodipropionic acid, 2,5-naphthalene-dicarboxylic acid and 2,6-naphthalenedicarboxylic acid. These dicarboxylic acid monomers may be used alone or as a combination of at least two dicarboxylic acid monomers. Among these monomers, phthalic acid, isophthalic acid and terephthalic acid may, for example, be used.

The diol may be chosen from aliphatic, alicyclic and aromatic diols. The diol used is, for example, chosen from ethylene glycol, diethylene glycol, triethylene glycol, 1,3-propanediol, cyclohexanedimethanol and 4-butanediol. Other polyols that may be used include glycerol, pentaerythritol, sorbitol and trimethylolpropane.

The polyesteramides may be obtained in a manner analogous to that of the polyesters, by polycondensation of diacids with diamines or amino alcohols. Diamines that may be used include, for example, ethylenediamine, hexamethylenediamine and meta- or para-phenylenediamine. An amino alcohol that may be used is, for example, monoethanolamine.

The polyester may also comprise at least one monomer bearing at least one —SO₃M group, wherein M is chosen from a hydrogen atom, an ammonium ion NH4+ and a metal ion such as an Na⁺, Li⁺, K⁺, Mg²⁺, Ca²⁺, Cu²⁺, Fe²⁺ or Fe³⁺ ion. A difunctional aromatic monomer comprising such an —SO₃M group may, for example, be used.

The aromatic nucleus of the difunctional aromatic monomer also comprising an —SO₃M group as described above may be chosen, for example, from benzene, naphthalene, anthracene, biphenyl, oxybiphenyl, sulfonylbiphenyl and methylenebiphenyl nuclei. Among the difunctional aromatic monomers also comprising an —SO₃M group, mention may be made, for example, of sulfoisophthalic acid, sulfoterephthalic acid, sulfophthalic acid, 4-sulfonaphthalene-2,7-dicarboxylic acid.

The copolymers used are, for example, those based on isophthalate/sulfoisophthalate, such as copolymers obtained by condensation of diethylene glycol, cyclohexanedimethanol, isophthalic acid and sulfoisophthalic acid.

The polymer for the film forming particles may also be a liposoluble polymer. Examples of the liposoluble polymer that may be mentioned include copolymers of a vinyl ester (wherein the vinyl group is directly linked to the oxygen atom of the ester group and the vinyl ester comprises a radical chosen from saturated, linear or branched hydrocarbon-based radicals of 1 to 19 carbon atoms, linked to the carbonyl of the ester group) and of at least one other monomer, which may be a vinyl ester (different from the vinyl ester already present), an a-olefin (comprising from 8 to 28 carbon atoms), an alkyl vinyl ether (the alkyl group of which comprises from 2 to 18 carbon atoms) or an allylic or methallylic ester (comprising a radical chosen from saturated, linear or branched hydrocarbon-based radicals of 1 to 19 carbon atoms, linked to the carbonyl of the ester group).

These copolymers may be crosslinked using crosslinking agents that may be either of the vinylic type or of the allylic or methallylic type, such as tetraallyloxyethane, divinylbenzene, divinyl octanedioate, divinyl dodecanedioate and divinyl octadecanedioate.

Examples of these copolymers which may be mentioned include the following copolymers: vinyl acetate/allyl stearate, vinyl acetate/vinyl laurate, vinyl acetate/vinyl stearate, vinyl acetate/octadecene, vinyl acetate/octadecyl vinyl ether, vinyl propionate/allyl laurate, vinyl propionate/vinyl laurate, vinyl stearate/1-octadecene, vinyl acetate/1-dodecene, vinyl stearate/ethyl vinyl ether, vinyl propionate/cetyl vinyl ether, vinyl stearate/allyl acetate, vinyl 2,2-dimethyloctanoate/vinyl laurate, allyl 2,2-dimethylpentanoate/vinyl laurate, vinyl dimethylpropionate/vinyl stearate, allyl dimethylpropionate/vinyl stearate, vinyl propionate/vinyl stearate, crosslinked with 0.2% divinylbenzene, vinyl dimethylpropionate/vinyl laurate, crosslinked with 0.2% divinylbenzene, vinyl acetate/octadecyl vinyl ether, crosslinked with 0.2% tetraallyloxyethane, vinyl acetate/allyl stearate, crosslinked with 0.2% divinylbenzene, vinyl acetate/1-octadecene, crosslinked with 0.2% divinylbenzene, and allyl propionate/allyl stearate, crosslinked with 0.2% divinylbenzene.

Further examples of the liposoluble film-forming polymers include liposoluble copolymers, such as those resulting from the copolymerization of vinyl esters comprising from 9 to 22 carbon atoms or of alkyl acrylates or methacrylates, wherein the alkyl radicals comprise from 10 to 20 carbon atoms. Such liposoluble copolymers may be chosen, for example, from polyvinyl stearate, polyvinyl stearate crosslinked with the aid of divinylbenzene, of diallyl ether or of diallyl phthalate copolymers, polystearyl (meth)acrylate, polyvinyl laurate and polylauryl (meth)acrylate copolymers, it being possible for these poly(meth)acrylates to be crosslinked with the aid of ethylene glycol dimethacrylate or tetraethylene glycol dimethacrylate. The liposoluble copolymers described above are known and are described, for example, in French patent application FR-A-2 232 303; they may have a weight-average molecular weight ranging, for example, from 2,000 to 500,000 such as from 4,000 to 200,000.

Among the liposoluble film-forming polymers which may be used herein, mention may also be made, for example, of polyalkylenes such as copolymers of C₂-C₂₀ alkenes, such as polybutene, alkylcelluloses with a linear or branched, saturated or unsaturated C₁-C₈ alkyl radical, for instance ethylcellulose and propylcellulose, copolymers of vinylpyrrolidone (VP) such as copolymers of vinylpyrrolidone and of C₂-C₄₀ alkene such as C₃-C₂₀ alkene. Among the VP copolymers which may be used herein, mention may be made, for example, of the copolymers of VP/vinyl acetate, VP/ethyl methacrylate, butylated polyvinylpyrrolidone (PVP), VP/ethyl methacrylate/methacrylic acid, VP/eicosene, VP/hexadecene, VP/triacontene, VP/styrene or VP/acrylic acid/lauryl methacrylate.

Specific examples of aqueous dispersions of film-forming particles which may be used are the acrylic dispersions sold under the names “Neocryl XK-90®”, “Neocryl A-1070®”, “Neocryl A-1090®”, “Neocryl BT-62®”, “Neocryl A-1079®” and “Neocryl A-523®” by the company Avecia-Neoresins, “Dow Latex 432®” by the company Dow Chemical, “Daitosol 5000 AD®” or “Daitosol 5000 SJ” by the company Daito Kasey Kogyo; the aqueous dispersions of polyurethane sold under the names “Neorez R-981®” and “Neorez R-974®” by the company Avecia-Neoresins, “Avalure UR-405®”, “Avalure UR-410®”, “Avalure UR-425®”, “Avalure UR-450®”, “Sancure 875®”, “Sancure 861®”, “Sancure 878®” and “Sancure 2060®” by the company Goodrich, “Impranil 85®” by the company Bayer and “Aquamere H-151®” by the company Hydromer; vinyl dispersions, for instance “Mexomer PAM” and also acrylic dispersions in isododecane, for instance “Mexomer PAP” by the company Chimex.

Further specific examples of latex polymers for use in the present invention further include a mixture of two copolymers of ethylene/acrylic acid and styrene acrylates (Syntran®PC 5288), ethylhexyl acrylate/hema copolymer (and) acrylates/diethylaminoethyl methacrylate/ethylhexyl acrylate copolymer (Syntran®PC 5775), styrene/acrylates/ammonium methacrylate copolymer (Syntran®5760, Syntran®5009, Syntran®PC5620), polyacrylate-21 (and) acrylates/dimethylaminoethyl methacrylate copolymer (Syntran®PC5100, Syntran®PC5776, Eudragit®E 100, Jurymer ET-410C), styrene/acrylates/ammonium methacrylate copolymer (Syntran®5009 CG), olefin/acrylate grafted polymer (and) sodium laureth sulfate (and C12-15 SEC-pareth 15 (Syntran®EX108), acrylates copolymer (Aculyn®33A Polymer, Avalure®Ace 210/120/315 Acrylic Copolymer, Carbopol® Aqua SF-1 Polymer, Coatex®Co 633, Eliclear®380/700/4U, Eudragit® L 100, Joncryl®85, Luviflex®Soft), acrylates/ethylhexyl acrylate copolymer. The Syntran® polymers are commercially available from the supplier Interpolymer Corp.

Preferably, the film forming particles comprise at least one hydrophilic side chain such as a side chain containing an OH group such as an acidic group or a hydroxyl group and/or a side chain containing a sulfonate group and/or a side chain containing an amino group Particularly preferred hydrophilic side chains include, but are not limited to, acrylic acid groups, methacrylic acid groups, and mixtures thereof.

Preferably, the film forming particles are present in the emulsions of the present invention in an amount sufficient to form a film upon a substrate to which it has been applied (for example, skin or eyelashes). When the film forming particles are in the form of a commercial product containing the film forming particles in aqueous dispersion, the amount of active material (that is, film forming particles) within the aqueous dispersion is sufficient to form a film upon a substrate to which it has been applied. Preferably, the film forming particles are present in the emulsions of the present invention in amounts of active material generally ranging from about 0.5% to 10%, preferably from about 0.75% to about 7.5%, and more preferably from about 1% to about 5%, by weight, based on the total weight of the composition, including all ranges and subranges in between.

Hydrophobic Filler

In accordance with the present invention, compositions (that is, emulsions) comprising at least one hydrophobic filler are provided. The filler itself may be hydrophobic itself, or the filler may have a hydrophobic coating.

Hydrophobicity can be determined by measuring the water contact angle of the filler and/or coating of the filler as is known in the art. For a material to be “hydrophobic,” the water contact angle is generally greater than 70 degrees. In accordance with preferred embodiments of the present invention, the at least one hydrophobic filler has associated with it and/or its coating a water contact angle of 80 to 150 degrees, including all ranges and subranges therebetween. Preferably, the water contact angle is between 80 and 120 degrees. Most preferably, the water contact angle is between 120 and 150 degrees.

The at least one hydrophobic filler can be colorless or white, inorganic or organic, and of any physical shape (platelet, spherical or oblong), and of any crystallographic form (for example sheet, cubic, hexagonal, orthorhombic and the like). The fillers can also be porous or nonporous.

Typical fillers may be lamellar or non-lamellar, inorganic or organic particles. Representative, non-limiting examples of these ingredients include mica, silica, kaolin, iron oxides, titanium dioxide, polyamide powders, poly-alanine powders, polyethylene powders, tetrafluoroethylene polymer powders, for instance polytetrafluoroethylene(Teflon®), lauroyllysine, silicon powders, starch, boron nitride, polymeric powders, polymethyl methacrylate particles and silicone resin microbeads (for example, Tospearls® from Toshiba), precipitated calcium carbonate, perlite, magnesium carbonate, magnesium hydrocarbonate, hydroxyapatite, hollow silica microspheres (Silica Beads® from Maprecos), glass or ceramic microcapsules.

Useful polymeric fillers can include, for example, lamellar or nonlamellar, colorless or white polymeric particles. In certain embodiments, the polymeric filler may be chosen from polyamide powders, such as Nylon® or Orgasol® powders from Arkema; cellulose poly-β-alanine and polyethylene powders; tetrafluoroethylene polymer powders, such as Teflon® powders; lauroyllysine; polymeric hollow microspheres such as those of polyvinylidene chloride/acrylonitrile, for instance Expancel® from Nobel Industries; acrylic powders such as acrylonitrile/methyl methacrylate/vinylidene chloride copolymer (Expancel 551, Akzo Nobel), and Polytrap® powders from Dow Corning; silicone resin microbeads, such as Tospearls® from Toshiba; elastomeric polyorganosiloxane particles, such as those obtained by polymerization of organopolysiloxane having at least two hydrogen atoms each bonded to a silicon atom and of an organopolysiloxane comprising at least two ethylenically unsaturated groups, for instance, two vinyl groups, in the presence of a platinum catalyst; and metal soaps derived from organic carboxylic acids comprising from 8 to 22 carbon atoms, such as from 12 to 18 carbon atoms, for example, zinc stearate, magnesium stearate, lithium stearate, zinc laurate, and magnesium myristate.

Silicone elastomer powders are also useful polymeric fillers. These powders include, but are not limited to, dipehnyl dimethicone/vinyl diphenyl dimethicone/silsesquioxane crosspolymer (e.g. KSP 300 from Shin Etsu), vinyl dimethicone/methicone silsesquioxane crosspolymer (e.g KSP 100, Shin Etsu), methylsilanol/silicate crosspolymer, and the powders sold under the names Trefil® Powder E-505C and Trefil® Powder E-506C by Dow Corning.

Acrylic polymer powders may also be used as fillers. Such powders include methacrylate polymers, for example methyl methacrylate/glycol dimethacrylate crosspolymer, methyl methacrylate crosspolymer, polymethyl methacrylate powders, polymethyl methacrylate/ethylene glycol dimethacrylate powders, polyallyl methacrylate/ethylene glycol dimethacrylate powders, and ethylene glycol dimethacrylate/lauryl methacrylate copolymer powders, and mixtures thereof.

Commercial examples of acrylic polymer powder products include methacrylate polymers such as polymethyl methacrylate powders sold under the name Covabead® LH85 by Wacker, DSPCS-12 series and SPCAT-12 from Kobo, and Poly-Pore 200 series from Amcol, and Techpolymer MBP-8 (methyl methacrylated crosspolymer), from Sekisui Plastics; the polymethyl methacrylate/ethylene glycol dimethacrylate powders sold under the names Microsponge® 5640 Skin Oil Adsorber (methyl methacrylate/glycol dimethacrylate crosspolymer by Dow Corning) and Ganzpearl® GMP-0820 by Ganz Chemical; the polyallyl methacrylate/ethylene glycol dimethacrylate powders sold under the name Poly-Pore® L200 and Poly-Pore® E200 by Amcol; and acrylic acid copolymers available from Dow Corning/Enhanced Derm Technologies under the name Polytrap® (for example ethylene glycol dimethacrylate/lauryl methacrylate copolymer powder, sold under the name Polytrap® 6603). Fibers can also be useful as hydrophobic fillers in accordance with the present invention. “Fiber” should be understood as meaning an object with a length L with a diameter D such that L is greater than D and preferably much greater than D, D being the diameter of the circle in which the cross section of the fibre appears. In particular, the ratio L/D (or aspect ratio) is chosen in the range extending from 3.5 to 2500, preferably from 5 to 500, and better still from 5 to 150.

The fibers which can be used in accordance with the present invention can be fibers of synthetic or natural, inorganic or organic origin, and they can be flexible or stiff. They can be short or long, individual or organized, for example braided. They can have any shape and can in particular have a circular or polygonal (square, hexagonal or octagonal) cross section, according to the specific application envisaged.

In particular, the fibers may have a length ranging from 1 μm to 10 mm, preferably from 0.1 mm to 5 mm and preferably from 0.1 mm to 3 mm Preferably, they have a cross section included within a circle with a diameter ranging from 2 nm to 500 μm, preferably ranging from 100 nm to 100 μm. The weight of the fibers is often given in denier or decitex.

Examples of fiber materials include, but are not limited to, those used in the manufacture of textiles such as silk, cotton, bamboo, wool or flax, cellulose fibers extracted, for example, from wood, vegetables or algae, fibers of polyamide (Nylon®, in particular under the names Nylon 6=Polyamide 6; Nylon 6,6 or Nylon 66=Polyamide 6,6; Nylon 12=Polyamide 12), rayon, viscose, acetate in particular rayon acetate, cellulose acetate or silk acetate, poly(p-phenylene terephthalamide) or acrylic polymer, in particular poly(methyl methacrylate) or poly(2-hydroxyethyl methacrylate), fibers of polyolefin and in particular of polyethylene or polypropylene, fibers of glass, silica, carbon, in particular in the graphite form, polytetrafluoroethylene (such as Teflon®), insoluble collagen, polyesters, poly(vinyl chloride), poly(vinylidene chloride), poly(vinyl alcohol), polyacrylonitrile, chitosan, polyurethane or poly(ethylene phthalate), fibers formed from a blend of polymers such as those mentioned above, for example polyamide/polyester fibers, and the blends of these fibers.

A “hydrophobic clay” or “lipophilic clay” means a clay that is capable of swelling in a lipophilic medium; this clay swells in the medium and thus forms a colloidal dispersion. Specific examples of hydrophobic clays or hydrophobically-modified clays include, but are not limited to, clays such as modified magnesium silicate (Bentone Gel VS38 from Rheox), and hectorites modified with a C₁₀ to C₂₂ fatty-acid ammonium chloride, for instance hectorite modified with distearyldimethylammonium chloride (CTFA name: disteardimonium hectorite) sold under the name Bentone 38 CE by the company Rheox or Bentone 38V® by the company Elementis.

Suitable hydrophobically-modified silicas include, but are not limited to, silicas such as pyrogenic silica optionally with hydrophobic surface treatment whose particle size is less than 1 micron, preferably less than 500 nm, preferably less than 100 nm, preferably from 5 nm to 30 nm, including all ranges and subranges therebetween. It is in fact possible to modify the surface of silica chemically, by a chemical reaction producing a decrease in the number of silanol groups present on the surface of the silica. The silanol groups can notably be replaced with hydrophobic groups: a hydrophobic silica is then obtained. The hydrophobic groups can be:

trimethylsiloxyl groups, which are notably obtained by treatment of pyrogenic silica in the presence of hexamethyldisilazane. Silicas treated in this way are called “Silica silylate” according to the CTFA (6th edition, 1995). They are for example marketed under the references “AEROSIL R812®” by the company Degussa, “CAB-O-SIL TS-530®” by the company Cabot;

dimethylsilyloxyl or polydimethylsiloxane groups, which are notably obtained by treatment of pyrogenic silica in the presence of polydimethylsiloxane or dimethyldichlorosilane. Silicas treated in this way are called “Silica dimethyl silylate” according to the CTFA (6th edition, 1995). They are for example marketed under the references “AEROSIL R972®”, “AEROSIL R974®” by the company Degussa, “CAB-O-SIL TS-610®”, “CAB-O-SIL TS-720®” by the company Cabot.

Preferably, the hydrophobic filler is present in the emulsions of the present invention in amounts of active material sufficient to reduce tackiness of the film formed by the emulsion after application, amounts generally ranging from about 0.1% to about 5%, preferably from about 0.25% to about 2.5%%, and preferably from 0.5 to about 1.5%, by weight, based on the total weight of the emulsion, including all ranges and subranges in between.

Preferably, film forming particles (active material) and hydrophobic filler are present in the emulsions of the present invention in a film forming particles to hydrophobic filler weight ratio of from 5:1 to1:5, preferably from 3:1 to 1:3, and preferably from 2:1 to 1:2, including all ranges and subranges therebetween.

Reaction Product

Preferably, a reaction product of at least one dispersion of film forming particles in aqueous phase and at least one hydrophobic filler is formed in the emulsions of the present invention.

Although not wishing to be bound by any particular theory, it is believed that when the film forming particles possess at least one hydrophilic side chain such as (meth)acrylic acid, the side chain can interact with fillers of significant hydrophobicity/polarity, and that such interaction surprisingly improves cosmetic properties such as long wearing properties. So, for example, it is believed that the film forming particles possessing at least one hydrophilic side chain such as (meth)acrylic acid interacts with the hydrophobic fillers by hydrophobic interactions van der Waals forces and/or electrostatic forces. Such interactions result in a formation of a filler/particle “composite” which forms a robust yet flexible film on the skin after application. The film has excellent wear properties including good transfer-resistance, good crease resistance, good comfort and easy removability.

Oil Phase

According to preferred embodiments of the present invention, the emulsions of the present invention may optionally further comprise at least one oil. “Oil” means any non-aqueous medium which is liquid at ambient temperature (25° C.) and atmospheric pressure (760 mm Hg).

Suitable oils include volatile and/or non-volatile oils. Such oils can be any acceptable oil including but not limited to silicone oils and/or hydrocarbon oils.

According to preferred embodiments, the composition of the present invention preferably comprise one or more volatile silicone oils. Examples of such volatile silicone oils include linear or cyclic silicone oils having a viscosity at room temperature less than or equal to 6 cSt and having from 2 to 7 silicon atoms, these silicones being optionally substituted with alkyl or alkoxy groups of 1 to 10 carbon atoms. Specific oils that may be used in the invention include octamethyltetrasiloxane, decamethylcyclopentasiloxane, dodecamethylcyclohexasiloxane, heptamethyloctyltrisiloxane, hexamethyldisiloxane, decamethyltetrasiloxane, dodecamethylpentasiloxane and their mixtures. Other volatile oils which may be used include KF 96A of 6 cSt viscosity, a commercial product from Shin Etsu having a flash point of 94° C. Preferably, the volatile silicone oils have a flash point of at least 40° C.

Non-limiting examples of volatile silicone oils are listed in Table 1 below.

TABLE 1

| | Flash Point | Viscosity |
|---|---|---|
| Compound | (° C.) | (cSt) |
| Octyltrimethicone | 93 | 1.2 |
| Hexyltrimethicone | 79 | 1.2 |
| Decamethylcyclopentasiloxane | 72 | 4.2 |
| (cyclopentasiloxane or D5) | | |
| Octamethylcyclotetrasiloxane | 55 | 2.5 |
| (cyclotetradimethylsiloxane or D4) | | |
| Dodecamethylcyclohexasiloxane (D6) | 93 | 7 |
| Decamethyltetrasiloxane(L4) | 63 | 1.7 |
| KF-96 A from Shin Etsu | 94 | 6 |
| PDMS (polydimethylsiloxane) DC 200 | 56 | 1.5 |
| (1.5 cSt) from Dow Corning | | |
| PDMS DC 200 (2 cSt) from Dow Corning | 87 | 2 |

Further, a volatile linear silicone oil may be employed in the present invention. Suitable volatile linear silicone oils include those described in U.S. Pat. No. 6,338,839 and WO03/042221, the contents of which are incorporated herein by reference. In one embodiment the volatile linear silicone oil is decamethyltetrasiloxane. In another embodiment, the decamethyltetrasiloxane is further combined with another solvent that is more volatile than decamethyltetrasiloxane.

According to preferred embodiments, the composition of the present invention preferably comprises one or more non-silicone volatile oils and may be selected from volatile hydrocarbon oils, volatile esters and volatile ethers. Examples of such volatile non-silicone oils include, but are not limited to, volatile hydrocarbon oils having from 8 to 16 carbon atoms and their mixtures and in particular branched C₈ to C₁₆ alkanes such as C₈ to C₁₆ isoalkanes (also known as isoparaffins), isododecane, isodecane, and for example, the oils sold under the trade names of Isopar or Permethyl. Preferably, the volatile non-silicone oils have a flash point of at least 40° C.

Non-limiting examples of volatile non-silicone volatile oils are given in Table 2 below.

TABLE 2

| Compound | Flash Point (° C.) |
|---|---|
| Isododecane | 43 |
| Propylene glycol n-butyl ether | 60 |
| Ethyl 3-ethoxypropionate | 58 |
| Propylene glycol methylether acetate | 46 |
| Isopar L (isoparaffin C₁₁-C₁₃) | 62 |
| Isopar H (isoparaffin C₁₁-C₁₂) | 56 |

The volatility of the solvents/oils can be determined using the evaporation speed as set forth in U.S. Pat. No. 6,338,839, the contents of which are incorporated by reference herein.

According to preferred embodiments of the present invention, the composition comprises at least one non-volatile oil. Examples of non-volatile oils that may be used in the present invention include, but are not limited to, polar oils such as:

hydrocarbon-based plant oils with a high triglyceride content consisting of fatty acid esters of glycerol, the fatty acids of which may have varied chain lengths, these chains possibly being linear or branched, and saturated or unsaturated; these oils are especially wheat germ oil, corn oil, sunflower oil, karite butter, castor oil, sweet almond oil, macadamia oil, apricot oil, soybean oil, rapeseed oil, cottonseed oil, alfalfa oil, poppy oil, pumpkin oil, sesame seed oil, marrow oil, avocado oil, hazelnut oil, grape seed oil, blackcurrant seed oil, evening primrose oil, millet oil, barley oil, quinoa oil, olive oil, rye oil, safflower oil, candlenut oil, passion flower oil or musk rose oil; or caprylic/capric acid triglycerides, for instance those sold by the company Stearineries Dubois or those sold under the names Miglyol 810, 812 and 818 by the company Dynamit Nobel;

synthetic oils or esters of formula R₅COOR₆ in which R₅ represents a linear or branched higher fatty acid residue containing from 1 to 40 carbon atoms, including from 7 to 19 carbon atoms, and R₆ represents a branched hydrocarbon-based chain containing from 1 to 40 carbon atoms, including from 3 to 20 carbon atoms, with R₆+R₇≧10, such as, for example, Purcellin oil (cetostearyl octanoate), isononyl isononanoate, C₁₂ to C₁₅ alkyl benzoate, isopropyl myristate, 2-ethylhexyl palmitate, and octanoates, decanoates or ricinoleates of alcohols or of polyalcohols; hydroxylated esters, for instance isostearyl lactate or diisostearyl malate; and pentaerythritol esters;

synthetic ethers containing from 10 to 40 carbon atoms;

C₈ to C₂₆ fatty alcohols, for instance coleyl alcohol, cetyl alcohol, stearyl alcohol, and cetearly alcohol; and

mixtures thereof.

Further, examples of non-volatile oils that may be used in the present invention include, but are not limited to, non-polar oils such as branched and unbranched hydrocarbons and hydrocarbon waxes including polyolefins, in particular Vaseline (petrolatum), paraffin oil, squalane, squalene, hydrogenated polyisobutene, hydrogenated polydecene, polybutene, mineral oil, pentahydrosqualene, and mixtures thereof.

According to preferred embodiments of the present invention, the at least oil is a high viscosity oil which is a silicone oil and/or a hydrocarbon oil.

Suitable examples of such silicone oils include, but are not limited to, non-volatile silicone fluids such as, for example, polyalkyl (aryl) siloxanes. Suitable polyalkyl siloxanes include, but are not limited to, polydimethyl siloxanes, which have the CTFA designation dimethicone, polydiethyl siloxane, phenyl trimethicone, trimethyl pentaphenyl trisiloxane, phenyldimethicone, phenyltrimethylsiloxydiphenylsiloxane, diphenyldimethicone, and diphenylmethyldiphenyltrisiloxane and those siloxanes disclosed in U.S. patent application publication no. 2004/0126350, the entire disclosure of which is hereby incorporated by reference. Specific examples of suitable high viscosity silicone oils include, but are not limited to, 15 M 30 from PCR (500 cSt) or Belsil PDM 1000 (1 000 cSt) from Wacker and Dow Corning 200 (350 cSt) (the values in parenthesis represent viscosities at 25° C.).

Suitable examples of such hydrocarbon oils include, but are not limited to, fluids having a molecular mass of more than 500 g/mol, for example more than 600 g/mol, and for example more than 650 g/mol. By “hydrocarbon” compound, it is meant a compound comprising principally atoms of carbon and hydrogen and optionally one or more functional groups chosen from hydroxyl, ester, ether and carboxyl functions. These compounds are, according to one aspect, devoid of —Si—O— groups. Suitable examples of hydrocarbon fluids include, but are not limited to polybutylenes, such as Indopol H-100 (of molar mass or MM=965 g/mol), Indopol H-300 (MM=1 340 g/mol), and Indopol H-1500 (MM=2 160 g/mol), which are sold or manufactured by Amoco; hydrogenated polyisobutylenes, such as Panalane H-300 E, sold or manufactured by Amoco (M=1 340 g/mol), Viseal 20000 sold or manufactured by Synteal (MM=6 000 g/mol), and Rewopal PIB 1000, sold or manufactured by Witco (MM=1 000 g/mol); polydecenes and hydrogenated polydecenes, such as Puresyn 10 (MM=723 g/mol) and Puresyn 150 (MM=9 200 g/mol) sold or manufactured by Mobil Chemicals; esters such as linear fatty acid esters having a total carbon number ranging from 30 to 70, such as pentaerythrityl tetrapelargonate (MM=697.05 g/mol); hydroxy esters, such as diisostearyl malate (MM=639 g/mol); aromatic esters such as tridecyl trimellitate (MM=757.19 g/mol); esters of C24-C28 branched fatty acids or fatty alcohols, such as those described in EP-A-0 955 039, for example triisocetyl citrate (MM=856 g/mol), pentaerythrityl tetraisononanoate (MM=697.05 g/mol), glyceryl triisostearate (MM=891.51 g/mol), glyceryl 2-tridecyltetradecanoate (MM=1 143.98 g/mol), pentaerythrityl tetraisostearate (MM=1 202.02 g/mol), poly-2-glyceryl tetraisostearate (MM=1 232.04 g/mol) and pentaerythrityl 2-tetradecyltetradecanoate (MM=1 538.66 g/mol); and mixtures thereof. Suitable ester oils can also be described according to formula R₁COOR₂ in which R₁ represents a linear or branched higher fatty acid residue containing from 1 to 40 carbon atoms, including from 7 to 19 carbon atoms, and R₂ represents a branched hydrocarbon-based chain containing from 1 to 40 carbon atoms, including from 3 to 20 carbon atoms, with R₁+R₂≧10, such as, for example, Purcellin oil (cetostearyl octanoate), isononyl isononanoate, C₁₂ to C₁₅ alkyl benzoate, isopropyl myristate, 2-ethylhexyl palmitate, and octanoates, decanoates or ricinoleates of alcohols or of polyalcohols; hydroxylated esters, for instance isostearyl lactate or diisostearyl malate; and pentaerythritol esters. A particularly preferred ester is diisostearyl malate.

According to preferred embodiments, the at least one oil is present in the compositions of the present invention in an amount ranging from about 0.5% to about 50% by weight, more preferably from about 1% to about 45% by weight, and most preferably from about 5% to about 40% by weight, based on the total weight of the composition, including all ranges and subranges within these ranges.

According to preferred embodiments of the present invention, the emulsions of the present invention may further comprise at least one wax. Suitable examples of waxes that can be used in accordance with the present disclosure include those generally used in the cosmetics field: they include those of natural origin, such as beeswax, carnauba wax, candelilla wax, ouricoury wax, Japan wax, cork fibre wax or sugar cane wax, rice wax, montan wax, paraffin wax, lignite wax or microcrystalline wax, ceresin or ozokerite, and hydrogenated oils such as hydrogenated castor oil or jojoba oil; synthetic waxes such as the polyethylene waxes obtained from the polymerization or copolymerization of ethylene, and Fischer-Tropsch waxes, or else esters of fatty acids, such as octacosanyl stearate, glycerides which are concrete at 30° C., for example at 45° C., silicone waxes, such as alkyl- or alkoxydimethicones having an alkyl or alkoxy chain ranging from 10 to 45 carbon atoms, poly(di)methylsiloxane esters which are solid at 30° C. and whose ester chain comprising at least 10 carbon atoms, or else di(1,1,1-trimethylolpropane) tetrastearate, which is sold or manufactured by Heterene under the name HEST 2T-4S, and mixtures thereof.

If present, the wax or waxes may be present in an amount ranging from 0.5%to about 20% by weight relative to the total weight of the composition, for example from about 1% to about 15%, and for example from 3% to 13%, including all ranges and subranges therebetween.

Coloring Agents

According to preferred embodiments of the present invention, emulsions optionally further comprising at least one coloring agent are provided.

Preferably, the at least one coloring agent is preferably chosen from pigments, dyes, such as liposoluble dyes, nacreous pigments, and pearling agents.

Representative liposoluble dyes which may be used according to the present invention include Sudan Red, DC Red 17, DC Green 6, β-carotene, soybean oil, Sudan Brown, DC Yellow 11, DC Violet 2, DC Orange 5, annatto, and quinoline yellow. The liposoluble dyes, when present, generally have a concentration ranging up to 20% by weight of the total weight of the composition, such as from 0.0001% to 6%, including all ranges and subranges therebetween.

The nacreous pigments which may be used according to the present invention may be chosen from white nacreous pigments such as mica coated with titanium or with bismuth oxychloride, colored nacreous pigments such as titanium mica with iron oxides, titanium mica with ferric blue or chromium oxide, titanium mica with an organic pigment chosen from those mentioned above, and nacreous pigments based on bismuth oxychloride. The nacreous pigments, if present, be present in the composition in a concentration ranging up to 50% by weight of the total weight of the composition, such as from 0.1% to 20%, preferably from 0.1% to 15%, including all ranges and subranges therebetween.

The pigments, which may be used according to the present invention, may be chosen from white, colored, inorganic, organic, polymeric, nonpolymeric, coated and uncoated pigments. Representative examples of mineral pigments include titanium dioxide, optionally surface-treated, zirconium oxide, zinc oxide, cerium oxide, iron oxides, chromium oxides, manganese violet, ultramarine blue, chromium hydrate, and ferric blue. Representative examples of organic pigments include carbon black, pigments of D & C type, and lakes based on cochineal carmine, barium, strontium, calcium, and aluminum.

If present, the pigments may be present in the composition in a concentration ranging up to 50% by weight of the total weight of the composition, such as from 0.5% to 40%, and further such as from 2% to 30%, including all ranges and subranges therebetween. In the case of certain products, the pigments, including nacreous pigments, may, for example, represent up to 50% by weight of the composition.

In preferred embodiments, the composition of the present invention has a continuous aqueous phase. For example, the composition of the present invention is in the form of a water-in-oil-in-water emulsion or in the form of an oil-in-water emulsion (O/W).

Additional Additives

The composition of the invention can also comprise any additive usually used in the field under consideration. For example, non-hydrophobic fillers, dispersants such as poly(12-hydroxystearic acid), antioxidants, film forming agents, essential oils, sunscreens, preserving agents, fragrances, fillers, neutralizing agents, cosmetic and dermatological active agents such as, for example, emollients, moisturizers, vitamins, essential fatty acids, surfactants, silicone elastomers, pasty compounds, viscosity increasing agents such as waxes or liposoluble/lipodispersible polymers, and mixtures thereof can be added. A non-exhaustive listing of such ingredients can be found in U.S. patent application publication no. 2004/0170586, the entire contents of which is hereby incorporated by reference. Further examples of suitable additional components can be found in the other references which have been incorporated by reference in this application. Still further examples of such additional ingredients may be found in the International Cosmetic Ingredient Dictionary and Handbook (9th ed. 2002).

A person skilled in the art will take care to select the optional additional additives and/or the amount thereof such that the advantageous properties of the composition according to the invention are not, or are not substantially, adversely affected by the envisaged addition.

These substances may be selected variously by the person skilled in the art in order to prepare a composition which has the desired properties, for example, consistency or texture.

These additives may be present in the composition in a proportion from 0% to 99% (such as from 0.01% to 90%) relative to the total weight of the composition and further such as from 0.1% to 50% (if present), including all ranges and subranges therebetween.

Needless to say, the composition of the invention should be cosmetically or dermatologically acceptable, i.e., it should contain a non-toxic physiologically acceptable medium and should be able to be applied to the eyelashes of human beings.

According to preferred embodiments of the present invention, methods of treating, caring for and/or making up keratinous material such as skin, lips, eyes and eyelashes by applying compositions of the present invention to the keratinous material in an amount sufficient to treat, care for and/or make up the keratinous material are provided. Preferably, “making up” the keratin material includes applying at least one coloring agent to the keratin material in an amount sufficient to provide color to the keratin material.

According to yet other preferred embodiments, methods of enhancing the appearance of keratinous material by applying compositions of the present invention to the keratinous material in an amount sufficient to enhance the appearance of the keratinous material are provided.

In accordance with the preceding preferred embodiments, the compositions of the present invention are applied topically to the desired area of the keratin material in an amount sufficient to treat, care for and/or make up the keratinous material, or to enhance the appearance of keratinous material. The compositions may be applied to the desired area as needed, preferably once or twice daily, more preferably once daily and then preferably allowed to dry before subjecting to contact such as with clothing or other objects (for example, a topcoat). Preferably, the composition is allowed to dry for about 1 minute or less, more preferably for about 45 seconds or less. The composition is preferably applied to the desired area that is dry or has been dried prior to application, or to which a basecoat has been previously applied.

According to other embodiments of the present invention, methods of methods of reducing tackiness of a film formed by at least one dispersion of film forming particles in aqueous phase are provided. These methods comprise combining at least one hydrophobic filler with the at least one dispersion of film forming particles in aqueous phase in an amount sufficient to reduce the tackiness of a film formed by the at least one dispersion of film forming particles in aqueous phase.

According to such methods, preferably, the hydrophobic filler is combined in an amount sufficient to reduce tackiness of the film formed by the film forming particles upon application to a substrate (for example, skin or eyelashes). For example, preferably, the hydrophobic filler is present in an amount sufficient to reduce the work of adhesion for the film to less than about 0.5 g s, preferably less than about 0.4 g s, preferably less than about 0.3 g s and preferably less than about 0.2 g s.

According to such methods, preferably, film forming particles (active material) and hydrophobic filler are present in a film forming particles to hydrophobic filler weight ratio of from 5:1 to 1:5, preferably from 3:1 to 1:3, and preferably from 2:1 to 1:2, including all ranges and subranges therebetween.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention.

Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective measurements. The following examples are intended to illustrate the invention without limiting the scope as a result. The percentages are given on a weight basis.

### EXAMPLES

### Example 1

### Preparation of Emulsions

Emulsions of the present invention can be prepared according to the following protocol:

disperse aqueous phase (phase A) at 70° C.;

add slowly melted oil phase (phase B) to A;

homogenize A-B at 70° C. for 20 minutes;

cool A-B to 50° C.;

add phase C to A-B while homogenizing;

homogenize for 10 minutes;

add phase D while mixing;

mix A-D for 30 minutes;

cool A-D to room temperature;

add phase E while mixing; and

mix for 10 minutes.

### Example 2

### Invention Eve shadow

| Phase | INCI | concentration |
|---|---|---|
| A | WATER | 43.32 |
| | SODIUM HYALURONATE | 0.10 |
| | GLYCERIN | 5.00 |
| | PHENOXYETHANOL | 0.50 |
| | SODIUM BENZOATE | 0.30 |
| | ADENOSINE | 0.10 |
| | AMMONIUM | 0.70 |
| | ACRYLOYLDIMETHYLTAURATE/STEARETH- | |
| | 25 METHACRYLATE CROSSPOLYMER | |
| | AMMONIUM POLYACRYLOYLDIMETHYL | 0.30 |
| | TAURATE | |
| | SUCROSE STEARATE | 1.30 |
| | PEG-11 METHYL ETHER DIMETHICONE (and) | 0.50 |
| | PPG-5-BUTETH-5 | |
| B | HYDROGENATED POLYISOBUTENE | 4.30 |
| | BEESWAX | 2.00 |
| | SORBITAN SESQUIOLEATE | 0.50 |
| | PENTAERYTHRITYL | 2.00 |
| | TETRAETHYLHEXANOATE | |
| | CYCLOHEXASILOXANE | 9.00 |
| C | ETHYLENE/ACRYLIC ACID COPOLYMER | 8.00 |
| | (and) STYRENE/ACRYLATES COPOLYMER | |
| | (and) C11-15 PARETH-40 (and) C11-15 | |
| | PARETH-7 (and) SODIUM LAURETH-12 | |
| | SULFATE | |
| D | PERLITE | 0.50 |
| | POLYMETHYLSILSESQUIOXANE | 1.00 |
| | SILICA SILYLATE | 1.10 |
| | SILICA | 0.50 |
| | MICA (and) IRON OXIDES (and) IRON OXIDES | 4.45 |
| | (and) TITANIUM DIOXIDE | |
| | MICA (and) TITANIUM DIOXIDE | 5.16 |
| | MICA (and) TITANIUM DIOXIDE (and) IRON | 1.16 |
| | OXIDES | |
| | MICA (and) TITANIUM DIOXIDE (and) | 0.26 |
| | YELLOW 5 LAKE (and) TIN OXIDE | |
| | MICA (and) IRON OXIDES (and) TITANIUM | 4.95 |
| | DIOXIDE | |
| E | ALCOHOL DENAT. | 3.00 |
| | | 100.00 |

### Example 3

### Invention Mascara

| Phase | INCI NAME | Weight |
|---|---|---|
| A | WATER | 54.05 |
| | PHENOXYETHANOL | 0.50 |
| | SODIUM BENZOATE | 0.30 |
| | AMMONIUM ACRYLOYLDIMETHYLTAURATE/ | 0.70 |
| | STEARETH-25 METHACRYLATE CROSSPOLYMER | |
| | AMMONIUM POLYACRYLOYLDIMETHYL | 0.30 |
| | TAURATE | |
| | SUCROSE STEARATE | 1.30 |
| | PEG-11 METHYL ETHER DIMETHICONE (and) | 0.50 |
| | PPG-5-BUTETH-5 | |
| B | HYDROGENATED POLYISOBUTENE | 4.30 |
| | BEESWAX | 8.00 |
| | SORBITAN SESQUIOLEATE | 0.50 |
| | PENTAERYTHRITYL TETRAETHYLHEXANOATE | 2.00 |
| | CETYL ALCOHOL | 3.00 |
| C | ETHYLENE/ACRYLIC ACID COPOLYMER (and) | 15.00 |
| | STYRENE/ACRYLATES COPOLYMER (and) C11-15 | |
| | PARETH-40 (and) C11-15 PARETH-7 (and) SODIUM | |
| | LAURETH-12 SULFATE | |
| D | PERLITE | 0.50 |
| | SILICA SILYLATE | 1.10 |
| | SILICA | 0.50 |
| | BLACK IRON OXIDE | 4.45 |
| E | ALCOHOL DENAT. | 3.00 |
| | | 100.00 |

### Example 4

### Invention Eyeliner

| Phase | INCI NAME | Weight |
|---|---|---|
| A | WATER | 47.80 |
| | PHENOXYETHANOL | 0.80 |
| | SODIUM BENZOATE | 0.30 |
| | AMMONIUM ACRYLOYLDIMETHYLTAURATE/ | 0.70 |
| | STEARETH-25 METHACRYLATE CROSSPOLYMER | |
| | SUCROSE STEARATE | 1.30 |
| | PEG-11 METHYL ETHER DIMETHICONE (and) | 0.50 |
| | PPG-5-BUTETH-5 | |
| B | SORBITAN SESQUIOLEATE | 0.50 |
| | PENTAERYTHRITYL TETRAETHYLHEXANOATE | 2.00 |
| | CYCLOHEXASILOXANE | 9.00 |
| C | ETHYLENE/ACRYLIC ACID COPOLYMER (and) | 15.00 |
| | STYRENE/ACRYLATES COPOLYMER (and) C11-15 | |
| | PARETH-40 (and) C11-15 PARETH-7 (and) SODIUM | |
| | LAURETH-12 SULFATE | |
| D | PERLITE | 0.50 |
| | POLYMETHYLSILSESQUIOXANE | 1.00 |
| | SILICA SILYLATE | 1.10 |
| | SILICA | 0.50 |
| | IRON OXIDES | 16.00 |
| E | ALCOHOL DENAT. | 3.00 |
| | | 100.00 |

### Example 5

### Invention Foundation

| Phase | INCI NAME | Weight |
|---|---|---|
| A | WATER | 42.24 |
| | SODIUM HYALURONATE | 0.10 |
| | GLYCERIN | 5.00 |
| | PHENOXYETHANOL | 0.50 |
| | SODIUM BENZOATE | 0.30 |
| | ADENOSINE | 0.10 |
| | AMMONIUM ACRYLOYLDIMETHYLTAURATE/ | 0.70 |
| | STEARETH-25 METHACRYLATE CROSSPOLYMER | |
| | AMMONIUM POLYACRYLOYLDIMETHYL | 0.30 |
| | TAURATE | |
| | SUCROSE STEARATE | 1.30 |
| | PEG-11 METHYL ETHER DIMETHICONE (and) | 0.50 |
| | PPG-5-BUTETH-5 | |
| B | DIMETHICONE | 7.00 |
| | SORBITAN SESQUIOLEATE | 0.50 |
| | PENTAERYTHRITYL TETRAETHYLHEXANOATE | 2.00 |
| | CYCLOHEXASILOXANE | 9.00 |
| C | ETHYLENE/ACRYLIC ACID COPOLYMER (and) | 10.00 |
| | STYRENE/ACRYLATES COPOLYMER (and) C11-15 | |
| | PARETH-40 (and) C11-15 PARETH-7 (and) SODIUM | |
| | LAURETH-12 SULFATE | |
| D | POLYMETHYLSILSESQUIOXANE | 1.00 |
| | SILICA SILYLATE | 1.10 |
| | SILICA | 0.50 |
| | TITANIUM DIOXIDE | 11.96 |
| | IRON OXIDES | 2.10 |
| | IRON OXIDES | 0.60 |
| | IRON OXIDES | 0.20 |
| E | ALCOHOL DENAT. | 3.00 |
| | | 100.00 |

### Example 6

### Testing of Eyeshadow of Example 1

The properties of the eye shadow in example 1 were determined.

Panel Studies

Fourteen sensory panelists trained in the application and evaluation of eye shadow products participated in the study. The eye shadows were applied to the eye lids using a standard protocol for cream eye shadow finger application. Samples were rated using a 15-point universal intensity scale (0=none/15=a lot), using a standard ballot for eye shadow. The eye shadows were evaluated during/immediately after application to the eye lids, as well as after 6 and 8 hours of wear.

One sample was evaluated each day using a sequential monadic test design. The samples were presented to panelists in a randomized order.

Upon application, the invention eyeshadow had significantly more Adherence to Eyelid, Intensity, Evenness of Deposit, Opaqueness, Shine, enhancing of fine lines, heaviness than comparative compositions which contain film forming particles but which did not contain at least one hydrophobic filler. At both 6 and 8 hours of wear, the invention eyeshadow had significantly more amount of Product Remaining, Evenness of Product, Opaqueness, Caking, Color Intensity, Shine, enhancing of fine lines that the comparative compositions.

In Vitro Studies

The products were deposited in 3 mil wet film on glass slides using a drown down bar and left under ambient condition for 24 hours in order to dry. Then, sebum was deposited on the sample surface. The samples were exposed to sebum for 24 hours followed by scratching down the film surface by a plastic tip using same force in at least 10 spots on the surface. The film delamination then was evaluated visually.

The film of the new eyeshadow demonstrated a higher resistance to the scratching as compared to comparative compositions, showing a slight level of delamination without reviling the blister card surface. The comparative composition was significantly delaminated revealing the surface of the blister card.

### Example 6

### Comparative Testing

The following combinations of latex, film formers and/or fillers were compared to the invention compositions and found to possess inferior properties: Syntran® PC 5288 up to 15% (4% solid)

Syntran® PC 5288 up to 15% (4% solid) with silica at 2%

Syntran® PC 5288 up to 8% (2.1% solid) with film forming agent polyvynylpirrolidone (PVP) at 1%

Syntran® PC 5288 up to 8% (2.1% solid) with PVP at 1% and with silica at 1%

Syntran® PC 5288 up to 8% (2.1% solid) with PVP at 1% and with silica at 1% and nylon at 1%

Baycusan® C1004 (no hydrophilic side chain) up to 11% (4.5% solid)

Baycusan® C1004 up to 7% (2.9% solid) with nylon at 1%

Baycusan® C1004 up to 7% (2.9% solid) with nylon at 1% and with silica silylate at 1%

Joncryl® 77 (no hydrophilic side chain) at 11% (5% solid)

Joncryl® at 5.5% (2.5% solid) and Diatasol 500ad at 5.5% (2.5% solid)

Joncryl® at 11% (5% solid) with silica silylate at 1%

Silica silylate at 1.5% (no latex)

1. An emulsion composition comprising about 5% to about 40% of at least one oil, at least one dispersion of film forming particles comprising at least one hydrophilic side chain in aqueous phase and at least one hydrophobic filler, wherein the film forming particles and hydrophobic filler are present in a film forming particles to hydrophobic filler weight ratio of from 5:1 to 1:5.

2. The composition of claim 1, wherein hydrophobic filler is present in an amount of from about 0.1% to 5%, based on the total weight of the composition.

3. The composition of claim 1, wherein the film forming particles and hydrophobic filler are present in a film forming particles to hydrophobic filler weight ratio of from 3:1 to 1:3.

4. The composition of claim 1, wherein the composition further comprises at least one coloring agent.

5. (canceled)

6. The composition of claim 1, wherein the composition is a foundation.

7. The composition of claim 1, wherein the composition is an eyeshadow.

8. A method of making up skin or eyelashes comprising applying the composition of claim 1 to the skin or eyelashes.

9. A method of reducing tackiness of a film formed by at least one dispersion of film forming particles comprising at least one hydrophilic side chain in aqueous phase comprising combining at least one hydrophobic filler with at least one dispersion of film forming particles in aqueous phase in an emulsion composition comprising about 5% to about 40% of at least one oil in an amount sufficient to reduce the tackiness of a film formed by the at least one dispersion of film forming particles in aqueous phase, wherein the film forming particles and hydrophobic filler are combined in a film forming particles to hydrophobic filler weight ratio of from 5:1 to 1:5.

10. The method of claim 9, wherein hydrophobic filler is present in an amount of from about 0.1% to 5%, based on the total weight of the composition.

11. The method of claim 9, wherein the film forming particles and hydrophobic filler are combined in a film forming particles to hydrophobic filler weight ratio of from 3:1 to 1:3.

12. (canceled)

13. The method of claim 9, wherein the composition is an eyeshadow.

14. The method of claim 9, wherein the composition is a foundation.

15. The method of claim 9, wherein the hydrophobic filler is present in an amount sufficient to reduce the work of adhesion of the film formed by at least 0.5 g s.

16. The method of claim 9, wherein the composition further comprises at least one coloring agent.

17. The composition of claim 1, wherein the at least one hydrophobic filler is selected from the group consisting of silicone elastomer powders, hydrophobically-modified silicas, and mixtures thereof.

18. The composition of claim 1, wherein the at least one hydrophobic filler is selected from the group consisting of silica silylate, polymethylsilsesquioxane, and mixtures thereof.

19. The method of claim 9, wherein the at least one hydrophobic filler is selected from the group consisting of silicone elastomer powders, hydrophobically-modified silicas, and mixtures thereof.

20. The method of claim 9, wherein the at least one hydrophobic filler is selected from the group consisting of silica silylate, polymethylsilsesquioxane, and mixtures thereof.

21. The composition of claim 1, wherein about 5% to about 40% of at least one volatile oil is present.

22. The method of claim 9, wherein about 5% to about 40% of at least one volatile oil is present.
